(19) United States
(12) Patent Application Publication
(10) Pub. No.: US 2025/0235090 A1
(43) Pub. Date: Jul. 24, 2025

(54) ENDOSCOPE SYSTEM, IMAGE GENERATION DEVICE, AND IMAGE GENERATION METHOD

(71) Applicant: OLYMPUS MEDICAL SYSTEMS CORP., Tokyo (JP)

(72) Inventor: Nobuyoshi ASAOKA, Ageo-shi (JP)

(73) Assignee: OLYMPUS MEDICAL SYSTEMS CORP., Tokyo (JP)

(21) Appl. No.: 19/175,263

(22) Filed: Apr. 10, 2025

Related U.S. Application Data
(63) Continuation of application No. PCT/JP2022/037862, filed on Oct. 11, 2022.

(51) Int. Cl. A61B 1/045; A61B 1/00; A61B 1/06

(52) U.S. Cl. CPC A61B 1/045; A61B 1/000096; A61B 1/0005; A61B 1/0655

(57) ABSTRACT

An endoscope system includes an endoscope including an illumination unit irradiating a subject and an imaging unit imaging the subject, a control device generating a captured image by performing imaging signal processing based on an imaging parameter, and an image generation device. The control device generates a first captured image in which the subject irradiated with a light intensity equal to or less than an upper limit value is imaged and a second captured image in which the subject irradiated with a light intensity equal to or less than the upper limit value is imaged and which is brighter than the first captured image. The image generation device generates an estimated image obtained by estimating an image in which the subject irradiated with a light intensity greater than the upper limit value is imaged from the first captured image and the second captured image.

### CROSS-REFERENCE TO RELATED APPLICATIONS

Priority is claimed on PCT/JP2022/037862, filed Oct. 11, 2022, the entire content of which is incorporated herein by reference.

### TECHNICAL FIELD

The present disclosure relates to an endoscope system, an image generation device, and an image generation method.

### BACKGROUND ART

An endoscope system includes an illumination unit and an imaging unit provided at a distal end portion, irradiates a subject with illumination light from the illumination unit, and images the subject using the imaging unit. The captured image is preferably bright enough to see the subject in detail.

When a light intensity of illumination light emitted from the illumination unit is increased to make a captured image bright enough, an upper limit value of the light intensity of illumination light is determined such that the temperature of the distal end portion does not exceed an allowable range. On the other hand, when a captured image is made to be bright enough through imaging signal processing such as gain adjustment, there is a likelihood that noise due to the imaging signal processing will occur to exceed an allowable range.

In Japanese Unexamined Patent Application, First Publication No. 2020-116147 (hereinafter referred to as Patent Document 1), an endoscope system that curbs a light intensity of illumination light to be equal to or less than an upper limit value except when a still image is acquired and curbs an increase in temperature of a distal end portion of an endoscope is described.

In the endoscope system described in Patent Document 1, there is a likelihood that the temperature of the distal end portion of the endoscope will exceed an allowable range when a still image is acquired.

### SUMMARY

The present disclosure provides an endoscope system, an image generation device, and an image generation method that can provide a captured image which is bright enough while curbing an increase in temperature of a distal end portion of an endoscope even when a still image is acquired.

According to a first aspect of the present disclosure, there is provided an endoscope system including: an endoscope including an illumination unit irradiating a subject with illumination light and an imaging unit imaging the subject; a control device generating a captured image by performing imaging signal processing on an imaging signal acquired from the imaging unit based on an imaging parameter; and an image generation device, wherein the control device generates a first captured image in which the subject irradiated with a light intensity equal to or less than an upper limit value is imaged and a second captured image in which the subject irradiated with a light intensity equal to or less than the upper limit value is imaged and which is brighter than the first captured image, and the image generation device generates an estimated image obtained by estimating an image in which the subject irradiated with a light intensity greater than the upper limit value is imaged from the first captured image and the second captured image.

According to a second aspect of the present disclosure, there is provided an image generation device for acquiring a captured image which is generated by performing imaging signal processing based on an imaging parameter on an imaging signal acquired from an imaging unit imaging a subject, the image generation device performing: acquiring a first captured image in which the subject irradiated with a light intensity equal to or less than an upper limit value is imaged and a second captured image in which the subject irradiated with a light intensity equal to or less than the upper limit value is imaged and which is brighter than the first captured image; and generating an estimated image obtained by estimating an image in which the subject irradiated with a light intensity greater than the upper limit value is imaged from the first captured image and the second captured image.

According to a third aspect of the present disclosure, there is provided an image generation method including: acquiring a first captured image which is generated by performing imaging signal processing on an imaging signal acquired by imaging a subject irradiated with a light intensity equal to or less than an upper limit value and a second captured image which is generated by performing imaging signal processing on an imaging signal acquired by imaging the subject irradiated with a light intensity equal to or less than the upper limit value and which is brighter than the first captured image; and generating an estimated image obtained by estimating an image in which the subject irradiated with a light intensity greater than the upper limit value is imaged from the first captured image and the second captured image.

With the endoscope system, the image generation device, and the image generation method according to the present disclosure, it is possible to provide a captured image which is bright enough while curbing an increase in temperature of a distal end portion of an endoscope even when a still image is acquired.

### BRIEF DESCRIPTION OF DRAWINGS

FIG. 1 is a diagram illustrating an endoscope system according to a first embodiment.

FIG. 2 is a functional block diagram of the endoscope system.

FIG. 3 is a functional block diagram of an imaging control unit 21 of the endoscope system.

FIG. 4 is a diagram illustrating a hardware configuration of an image generation device of the endoscope system.

FIG. 5 is a functional block diagram of the image generation device.

FIG. 6 is a diagram illustrating a relationship between a first captured image and a second captured image.

FIG. 7 is a diagram illustrating generation of a trained model in the endoscope system.

FIG. 8 is a diagram illustrating training data that is used to generate the trained model.

FIG. 9 is a control flowchart in the endoscope system.

FIG. 10 is a diagram illustrating a modified example of the trained model.

FIG. 11 is a diagram illustrating another modified example of the trained model.

FIG. 12 is a diagram illustrating a moving image that is generated by an image generating unit.

FIG. 13 is a diagram illustrating a captured image that is generated by an imaging control unit and a still image that is generated by an image generating unit in an endoscope system according to a second embodiment.

FIG. 14 is a control flowchart in the endoscope system.

FIG. 15 is a functional block diagram of an endoscope system according to a third embodiment.

FIG. 16 is a diagram illustrating an image generating unit of the endoscope system.

FIG. 17 is a diagram illustrating a relationship between a first special-light captured image and a second special-light captured image.

FIG. 18 is a diagram illustrating a modified example of a trained model in the endoscope system.

FIG. 19 is a diagram illustrating generation of the trained model.

FIG. 20 is a diagram illustrating training data that is used to generate the trained model.

FIG. 21 is a diagram illustrating an image generating unit of an endoscope system according to a fourth embodiment.

FIG. 22 is a diagram illustrating generation of a trained model in the endoscope system.

### DESCRIPTION

### First Embodiment

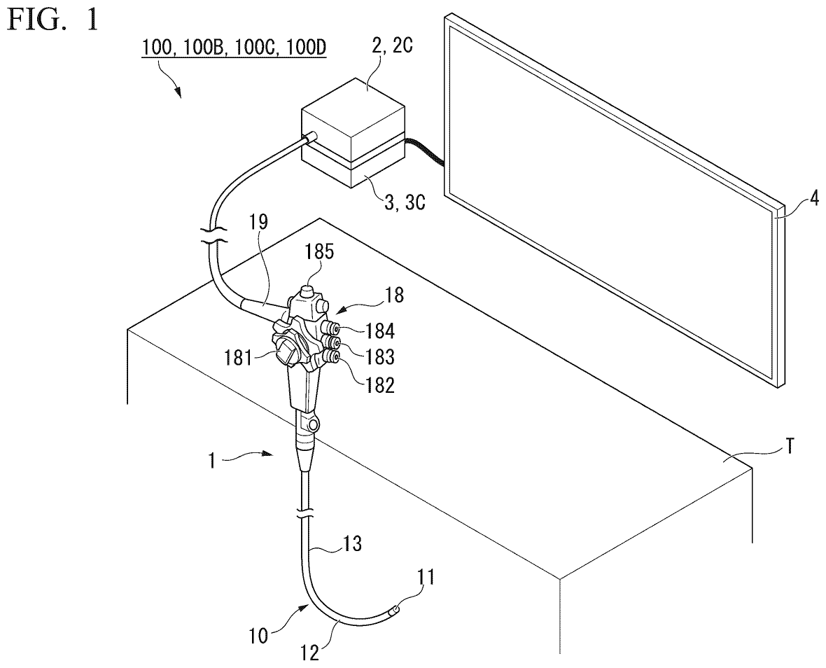

An endoscope system 100 according to a first embodiment of the present disclosure will be described below with reference to FIGS. 1 to 9.

### Endoscope System 100

FIG. 1 is a diagram illustrating the endoscope system 100.

The endoscope system (an image generation system) 100 includes an endoscope 1, a control device 2, an image generation device 3, and a display device 4. The control device 2 and the image generation device 3 may be a unified device (an image control device). The display device 4 is a device that displays an image generated by the control device 2 or the image generation device 3, various types of information on the endoscope system 100, and the like.

### Endoscope 1

The endoscope 1 is, for example, a device that is used to observe and treat an internal part of a patient laid on an operating table T. The endoscope 1 includes a thin and long insertion unit 10 which is inserted into a patient, an operation unit 18 that is connected to a proximal end of the insertion unit 10, and a universal cord 19 that extends from the operation unit 18.

The insertion unit 10 includes a distal end portion 11, a bendable bending portion 12, and a flexible tube portion 13 that is long and flexible. The distal end portion 11, the bending portion 12, and the flexible tube portion 13 are sequentially connected from the distal end. The flexible tube portion 13 is connected to the operation unit 18.

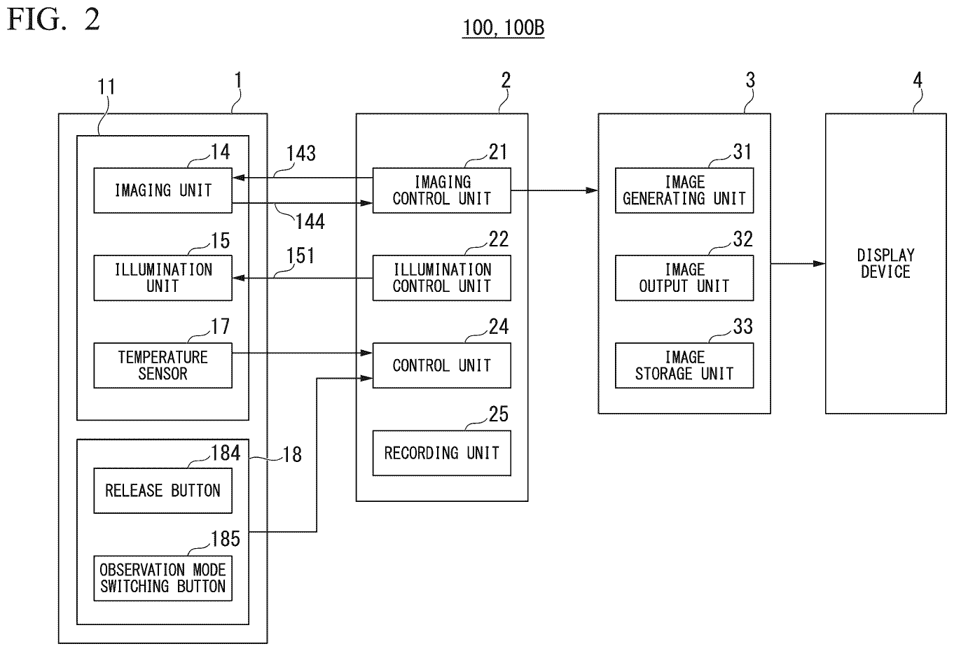

FIG. 2 is a functional block diagram of the endoscope system 100.

The distal end portion 11 includes an imaging unit 14, an illumination unit 15, and a temperature sensor 17.

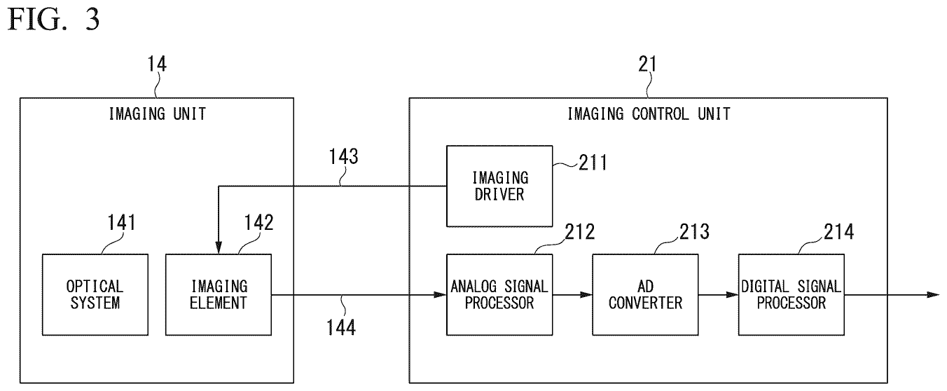

The imaging unit 14 includes an optical system 141 and an imaging element 142 such as a CCD image sensor or a CMOS image sensor (see FIG. 3). The imaging unit 14 images a subject based on an imaging parameter P transmitted from an imaging control unit 21 via an imaging control cable 143 and generates an imaging signal. The imaging signal is transmitted to the control device 2 via an imaging signal cable 144.

The illumination unit (white-light illumination unit) 15 irradiates a subject with illumination light (white light) transmitted by a ride guide 151. The ride guide 151 is inserted into the insertion unit 10, the operation unit 18, and a universal cord 19 and is connected to the control device 2. The illumination unit 15 may include a light source such as an LED and an optical element such as a fluorescent substance having a wavelength conversion function.

The temperature sensor 17 is a sensor that detects a temperature of the illumination unit 15. Examples of the temperature sensor 17 include a thermocouple, a thermistor, a thermosensitive resistor, a thermosensitive ferrite, and a thermally expanding thermometer. The detected temperature of the illumination unit 15 is acquired by the control device 2.

The operation unit 18 receives an operation on the endoscope 1. The operation unit 18 includes an ankle knob 181 controlling the bending portion 12, an air-supply/water-supply button 182, a suction button 183, a release button 184, and an observation mode switching button 185. Operations input to the air-supply/water-supply button 182, the suction button 183, the release button 184, and the observation mode switching button 185 are acquired by the control device 2. The release button 184 is a button that is pressed to input an operation of storing a captured image acquired from the imaging unit 14.

The universal cord 19 connects the endoscope 1 and the control device 2. The universal cord 19 is a cable into which the imaging control cable 143, the imaging signal cable 144, the ride guide 151, and the like are inserted.

### Control Device 2

The control device 2 is a device that controls the endoscope system 100 as a whole. The control device 2 includes an imaging control unit 21, an illumination control unit 22, a control unit 24, and a recording unit 25.

FIG. 3 is a functional block diagram of the imaging control unit 21.

The imaging control unit 21 performs imaging signal processing based on the image parameter P on an imaging signal acquired from the imaging unit 14 and generates a captured image (a captured video) D_{W}. The imaging control unit 21 includes an imaging driver 211, an analog signal processor 212, an AD converter 213, and a digital signal processor 214.

The imaging driver 211 drives the imaging element 142 of the imaging unit 14 based on the imaging parameter P from the control unit 24. The imaging driver 211 controls exposure of the imaging element 142.

The analog signal processor 212 performs analog signal processing including noise reduction and amplification on an imaging signal acquired from the imaging element of the imaging unit 14 based on the imaging parameters P (such as an analog gain and an ISO sensitivity) from the control unit 24.

The AD converter 213 converts the imaging signal on which the analog signal processing has been performed by the analog signal processor 212 to a digital signal (for example, RAW data) based on an instruction from the control unit 24.

The digital signal processor 214 processes the digital signal (for example, RAW data) on which conversion has been performed by the AD converter 213 based on the imaging parameter P from the control unit 24 and generates a captured image. The digital signal processor 214 performs digital signal processing for adjusting luminance, contrast, or the like on the captured image according to necessity.

The illumination control unit 22 includes a light source such as an LED and controls the light source based on the imaging parameter P (such as a light intensity) from the control unit 24 such that the light intensity of illumination light transmitted to the illumination unit 15 via the ride guide 151 is controlled.

The control unit 24 is a program-executable processing circuit (computer) including a processor and a program-readable memory. The control unit 24 controls the endoscope system 100 by executing an endoscope control program. The control unit 24 may include a dedicated circuit. The dedicated circuit is a processor separate from the processor of the control unit 24, a logic circuit mounted on an ASIC or an FPGA, or a combination thereof.

The control unit 24 controls the imaging control unit 21 and the illumination control unit 22 such that a captured image D_{W} is generated by designating an operation parameter including the imaging parameter P for the imaging control unit 21 and the illumination control unit 22. The control unit 24 issues an image generation instruction to the image generation device 3 or a display instruction to the display device 4.

The recording unit 25 is a nonvolatile recording medium storing the program or necessary data. The recording unit 25 is constituted, for example, by a writable nonvolatile memory such as a flexible disk, a magneto-optical disc, a ROM, or a flash memory, a portable medium such as a CD-ROM, or a storage device such as a hard disk incorporated into a computer system. The recording unit 25 may be a storage device or the like provided in a cloud server connected to the control device 2 via the Internet.

### Image Generation Device 3

The image generation device 3 is a device that generates a new estimated image E_{W} based on a captured image (a captured video) D_{W} output by the imaging control unit 21 of the control device 2 and displays the generated estimated image E_{W} on the display device 4. When the new estimated image E_{W} does not need to be generated, the image generation device 3 displays the captured image output by the imaging control unit 21 of the control device 2 on the display device 4.

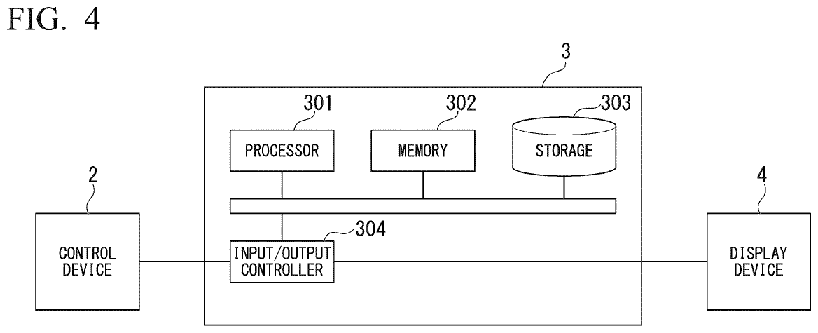

FIG. 4 is a diagram illustrating a hardware configuration of the image generation device 3.

The image generation device 3 includes a processor 301, a program-readable memory 302, a storage 303, and an input/output controller 304. The image generation device 3 is a program-executable computer. The functions of the image generation device 3 are realized by causing the processor 301 to execute a program. At least some functions of the image generation device 3 may be realized by a dedicated logic circuit mounted in an ASIC or an FPGA.

The storage 303 is a nonvolatile recording medium storing the program or necessary data. The storage 303 is constituted, for example, by a ROM or a hard disk. The program recorded on the storage 303 is read to the memory 302 and is executed by the processor 301.

The input/output controller 304 is connected to the control device 2, the display device 4, an input device (not illustrated), and a network device (not illustrated). The input/output controller 304 performs transmission and reception of data or transmission and reception of control signals with respect to a device connected thereto under the control of the processor 301.

The image generation device 3 is not limited to a unified hardware device. For example, the image generation device 3 may be configured by separating a part thereof as an independent hardware device and connecting the separated hardware device via a communication line. For example, the image generation device 3 may be a cloud system connecting the separated storage 303 via a communication line.

The image generation device 3 may further include a constituent other than the processor 301, the memory 302, the storage 303, and the input/output controller 304. For example, the image generation device 3 may further include an image operation unit performing some or all of image processing and image recognition processing. When the image operation unit is further included, the image generation device 3 can perform specific image processing or image recognition processing at a high speed. For example, the image generation device 3 may further include an inference operation unit performing some or all of inference processing of an estimated image E_{W} which will be described later. When the inference operation unit is further included, the image generation device 3 can perform inference processing of the estimated image E_{W} at a high speed. The image operation unit or the inference operation unit may be counted in an independent hardware device connected thereto via a communication line.

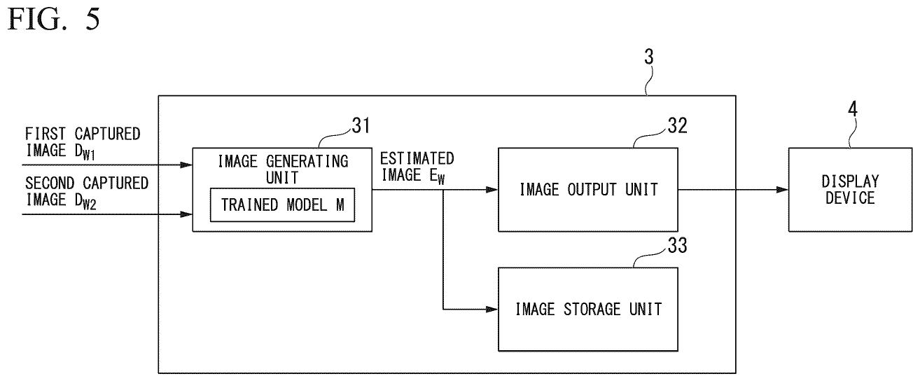

FIG. 5 is a functional block diagram of the image generation device 3.

The image generation device 3 includes an image generating unit 31, an image display unit 32, and an image storage unit 33 as functional blocks.

The image generating unit 31 generates an estimated image (a white-light estimated image) E_{W} from a first captured image (a first white-light captured image) D_{W1} and a second captured I mage (a second white-light captured image) D_{W2} input from the control device 2 based on a trained model M.

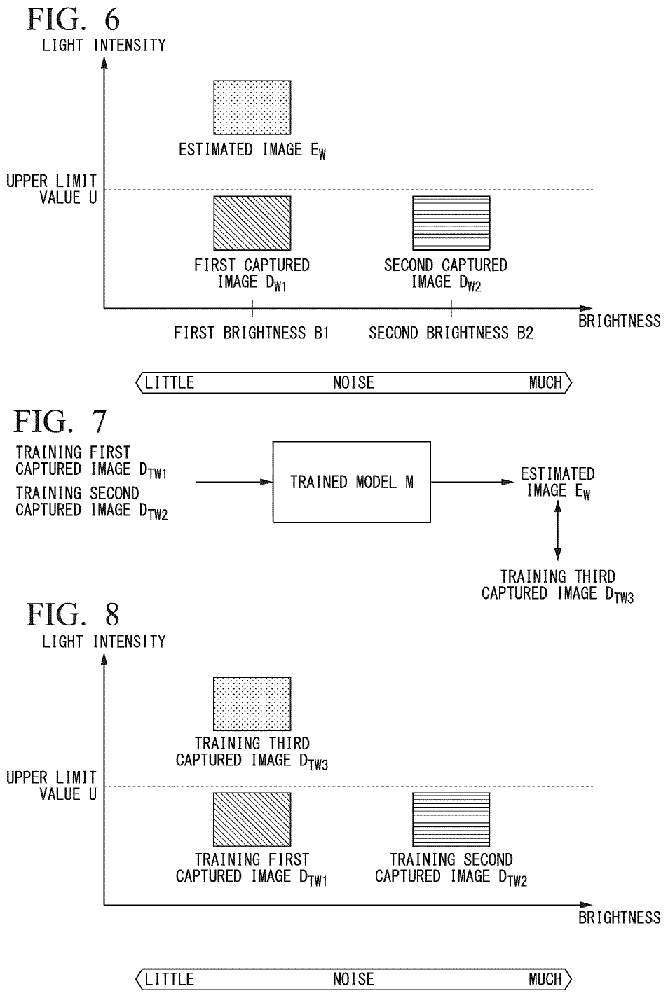

FIG. 6 is a diagram illustrating a relationship between the first captured image D_{W1} and the second captured image D_{W2}.

The first captured image D_{W1} is a captured image in which a subject irradiated with a light intensity equal to or less than an upper limit value U is imaged. The second captured image D_{W2} is a captured image in which the subject irradiated with a light intensity equal to or less than the upper limit value U is imaged and which has more noise and higher brightness than the first captured image D_{W1} through imaging signal processing (analog signal processing by the analog signal processor 212 and/or digital signal processing by the digital signal processor 214) based on the imaging parameter P. “Brightness of a captured image” is, for example, “brightness,” “luminance,” and “luminous intensity.” In the following description, the brightness of the first captured image D_{W1} is also referred to as “first brightness B1,” and the brightness of the second captured image D_{W2} is also referred to as “second brightness B2.”

The first captured image D_{W1} and the second captured image D_{W2} are captured images in which a subject irradiated substantially with the same light intensity is imaged. The first captured image D_{W1} and the second captured image D_{W2} do not have to be captured images in which a subject irradiated substantially with the same light intensity is imaged.

The first captured image D_{W1} and the second captured image D_{W2} are preferably captured images in which a subject irradiated with a light intensity equal to or less than the upper limit value U and close to the upper limit value U is imaged. By causing the light intensity to become closer to the upper limit value U, the first captured image D_{W1} and the second captured image D_{W2} are captured images which are brighter and which have less noise. As a result, it is possible to enhance estimation accuracy of the estimated image E_{W}. Particularly, by decreasing noise of the second captured image D_{W2}, it is possible to efficiently enhance estimation accuracy of the estimated image E_{W}.

The second captured image D_{W2} is a captured image which has been captured when a period T1 has elapsed after the first captured image D_{W1} has been captured. Since the period T1 is a very short period, the subject imaged in the first captured image D_{W1} and the second captured image D_{W2} is substantially in the same state. The order of imaging of the first captured image D_{W1} and the second captured image D_{W2} is not particularly limited.

The first captured image D_{W1} and the second captured image D_{W2} are input to a trained model M. In order to enhance the estimation accuracy of the estimated image E_{W} output from the trained model M, it is preferable that a difference between information included in the first captured image D_{W1} and information included in the second captured image D_{W2} be large. Accordingly, it is preferable that the imaging parameter P for generating the first captured image D_{W1} and the imaging parameter P for generating the second captured image D_{W2} be more different.

The imaging signal processing includes, for example, a process of amplifying an analog signal or a process of amplifying a digital signal. In this case, the gain of the imaging parameter P for generating the second captured image D_{W2} is greater than the gain of the imaging parameter P for generating the first captured image D_{W1}.

The process of amplifying an analog signal can be more suitably used to enhance the estimation accuracy of the estimated image E_{W} than the process of amplifying a digital signal. This is because the process of amplifying a digital signal is performed after AD conversion in which partial information of an imaging signal disappears and thus is disadvantageous in view of enhancement of the difference between information included in the first captured image D_{W1} and information included in the second captured image D_{W2}. On the other hand, the process of amplifying a digital signal can be more suitably performed to perform more various and flexible imaging signal processing than the process of amplifying an analog signal. This is because the process of amplifying a digital signal can generate the first captured image D_{W1} and the second captured image D_{W2}, for example, by performing imaging signal processing on one type of imaging signal acquired from the imaging unit 14 based on the gains of two types of imaging parameters P.

The estimated image E_{W} is an image which is generated based on a trained model M from the first captured image D_{W1} and the second captured image D_{W2} and which is obtained by estimating an image in which the subject irradiated with a light intensity greater than the upper limit value U.

The image display unit 32 outputs the estimated image E_{W} generated by the image generating unit 31 to the display device 4 such that the display device 4 displays the estimated image.

The image storage unit 33 records the estimated image E_{W} generated by the image generating unit 31. When the estimated image E_{W} does not need to be recorded, the image storage unit 33 is not necessary.

### Trained Model M

The trained model M is a machine learning model learning a relationship between an input image and an output image and is, for example, a machine learning model which is appropriate for generating an image such as a neural network, a simple perceptron, or a multilayer perceptron. The trained model M is generated through beforehand supervised learning based on training data. Generation of the trained model M may be performed by the image generation device 3 or may be performed using another computer having higher operation performance than that of the image generation device 3.

FIG. 7 is a diagram illustrating generation of a trained model M.

Training data is an image group including a training first captured image D_{TW1}, a training second captured image D_{TW2}, and a training third captured image D_{TW3} as a group of images. The training data preferably includes images captured under various conditions as many as possible.

The trained model M generates an estimated image E_{W} based on the training first captured image D_{TW1} and the training second captured image D_{TW2} which are input thereto. The trained model M learns parameters of a model M which has been trained such that a difference between the estimated image E_{W} which is output and the training third captured image D_{TW3} decreases.

FIG. 8 is a diagram illustrating training data which is used to generate a trained model M.

The training first captured image D_{TW1} is a first captured image D_{W1} prepared for training. The training second captured image D_{TW2} is a second captured image D_{W2} which is prepared for training.

The training third captured image D_{TW3} is a captured image in which a subject irradiated with a light intensity greater than the upper limit value U is actually imaged. The training third captured image D_{TW3} is an image in which noise generated through the imaging signal processing (the analog signal processing by the analog signal processor 212 and/or the digital signal processing by the digital signal processor 214) based on the imaging parameter P has the same degree as noise generated in the training first captured image D_{TW1} through the imaging signal processing. For example, the training third captured image D_{TW3} is an image for which imaging parameters P other than the light intensity are substantially the same as the imaging parameters P for the training first captured image D_{TW1}.

The training third captured image D_{TW3} is a captured image which is captured when a period T2 has elapsed after the training first captured image D_{TW1} and the training second captured image D_{TW2} have been captured. Since the period T2 is a very short period, the subject imaged in the training first captured image D_{TW1}, the training second captured image D_{TW2}, and the training third captured image D_{TW3} is substantially in the same state. The order of imaging of the training first captured image D_{TW1}, the training second captured image D_{TW2}, and the training third captured image D_{TW3} is not particularly limited. The training first captured image D_{TW1}, the training second captured image D_{TW2}, and the training third captured image D_{TW3} may be images in which a stationary subject is imaged. In this case, the subject imaged in the training first captured image D_{TW1}, the training second captured image D_{TW2}, and the training third captured image D_{TW3} are in the same state.

It is preferable that the brightness of the training first captured image D_{TW1} and the brightness of the training second captured image D_{TW2} be substantially equal to the first brightness B1 and the second brightness B2. This is because the trained model M can more efficiently learn the relationship between the first captured image D_{W1} and the second captured image D_{W2}.

The training second captured image D_{TW2} is preferably a captured image which is captured when a period Tl has elapsed after the training first captured image D_{TW1} has been captured. This is because the trained model M can more efficiently learn the relationship between the first captured image D_{W1} and the second captured image D_{W2}.

Through the aforementioned supervised learning using the training data, a trained model M having learned the relationship between the first captured image D_{W1} and the second captured image D_{W2} and the estimated image E_{W} is generated. Noise included in the estimated image E_{W} output from the trained model M to which the first captured image D_{W1} and the second captured image D_{W2} have been input has the same degree as noise generated in the first captured image D_{W1} through the imaging signal processing.

### Operations of Endoscope System 100

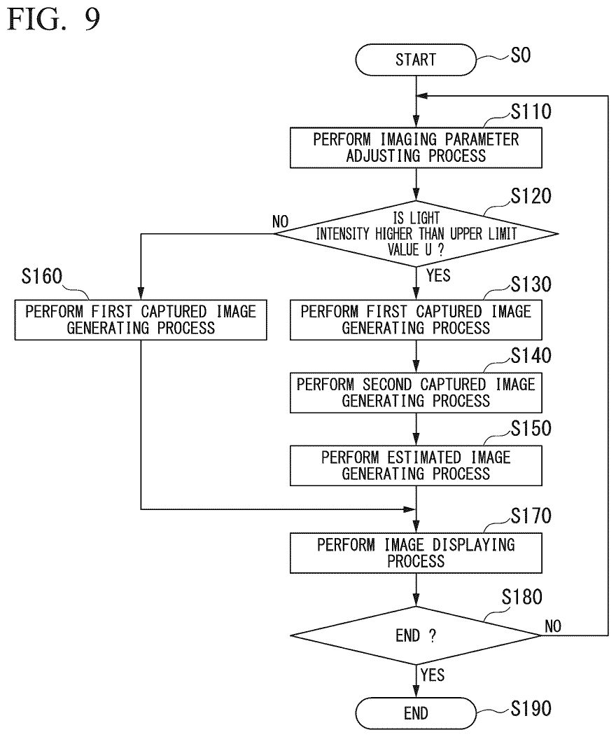

The operations (an image generation method) of the endoscope system 100 will be described below. The operations will be described with reference to a control flowchart of the endoscope system 100 illustrated in FIG. 9. When the control unit 24 of the control device 2 detects an operator's operation of pressing the release button 184, the endoscope system 100 performs Step S110.

### Step S110: Imaging Parameter Adjusting Step>

In Step S110, the control unit 24 of the control device 2 calculates imaging parameters P (which include a light intensity and an analog gain) which are optimal for imaging a subject based on a captured image captured by the imaging control unit. Known imaging adjustment techniques are used to calculate the optimal imaging parameters P. Then, the endoscope system 100 performs Step S120.

### Step S120: Light Intensity Determining Step

In Step S120, the control unit 24 of the control device 2 determines whether the calculated light intensity is greater than the upper limit value U. When the calculated light intensity is greater than the upper limit value U, the control unit 24 of the control device 2 performs Step S130. When the calculated light intensity is equal to or less than the upper limit value U, the endoscope system 100 performs Step S160.

The upper limit value U is not a fixed value but may be a value varying according to the temperature of the illumination unit 15 acquired by the temperature sensor 17 of the endoscope 1. For example, when the temperature of the illumination unit 15 is lower than a predetermined temperature, the control unit 24 may increase the upper limit value U. When the temperature of the illumination unit 15 is higher than the predetermined temperature, the control unit 24 may decrease the upper limit value U. When the endoscope 1 does not include the temperature sensor 17, the control unit 24 may use the temperature which is estimated from an integrated light intensity obtained by multiplying the light intensity by a light emission time.

### Step S130: First Captured Image Generating Step

In Step S130, the control unit 24 of the control device 2 sets the light intensity which is an imaging parameter P to be equal to or less than the upper limit value U and generates the first captured image D_{W1}. Then, the endoscope system 100 performs Step S140.

### Step S140: Second Captured Image Generating Step

In Step S140, the control unit 24 of the control device 2 changes the imaging parameters P other than the light intensity and generates the second captured image D_{W2} which has more noise and higher brightness than the first captured image D_{W1}. For example, the control unit 24 increases the analog gain which is an imaging parameter P and generates the second captured image D_{W2}. The control unit 24 of the control device 2 generates the second captured image D_{W2} when a period T1 has elapsed after the first captured image D_{W1} has been captured. Then, the endoscope system 100 performs Step S150.

### Step S150: Estimated Image Generating Step

In Step S150, the image generating unit 31 of the image generation device 3 generates an estimated image E_{W} from the first captured image D_{W1} and the second captured image D_{W2} input from the imaging control unit 21 of the control device 2 based on the trained model M. The image storage unit 33 records the estimated image E_{W} according to necessity. Then, the endoscope system 100 performs Step S170.

### Step S160: First Captured Image Generating Step

In Step S160, the control unit 24 and the imaging control unit 21 of the control device 2 generate a first captured image D_{W1}. Since the light intensity calculated in Step S110 is equal to or less than the upper limit value U, the control unit 24 of the control device 2 sets the light intensity to the calculated light intensity and generates the first captured image D_{W1}. Then, the endoscope system 100 performs Step S170.

### Step S170: Image Displaying Step

The image display unit 32 of the image generation device 3 displays the estimated image E_{W} generated in Step S150 or the first captured image D_{W1} captured in Step S160 on the display device 4. Then, the endoscope system 100 performs Step S180.

### Step S180: End Determining Step

In Step S180, the control unit 24 of the control device 2 determines whether an operation of pressing the release button 184 has been performed by an operator or the like. When an operation of pressing the release button 184 has been performed, the control unit 24 of the control device 2 performs Step S110 again.

With the endoscope system 100 according to the present embodiment, even when a still image is acquired, it is possible to provide a captured image which is bright enough while curbing an increase in temperature of the distal end portion 11 of the endoscope 1. Specifically, since the endoscope system 100 uses only the first captured image D_{W1} and the second captured image D_{W2} in which a subject irradiated with a light intensity equal to or less than the upper limit value U is imaged, it is possible to curb a light intensity of illumination light emitted from the illumination unit 15 to be equal to or less than the upper limit value U and to curb the temperature of the distal end portion 11 of the endoscope 1 to be equal to or less than a predetermined temperature. On the other hand, the image generation device 3 of the endoscope system 100 can infer an estimated image E_{W} obtained by estimating an image in which the subject irradiated with a light intensity greater than the upper limit value U is imaged from the first captured image D_{W1} and the second captured image D_{W2} in which the subject irradiated with a light intensity equal to or less than the upper limit value U is imaged. That is, with the endoscope system 100, it is possible to curb a light intensity of illumination light emitted from the illumination unit 15 to be equal to or less than the upper limit value U and to present to a user an estimated image E_{W} which is brighter than the first captured image D_{W1} and in which noise included in the image has the same degree as noise generated in the first captured image D_{W1} through the imaging signal processing.

While the first embodiment of the present disclosure has been described above in detail with reference to the drawings, any specific configuration is not limited to this embodiment and includes design modification or the like without departing from the gist of the present disclosure. The constituents described in the aforementioned embodiment and the modified examples can be appropriately combined.

### MODIFIED EXAMPLE 1

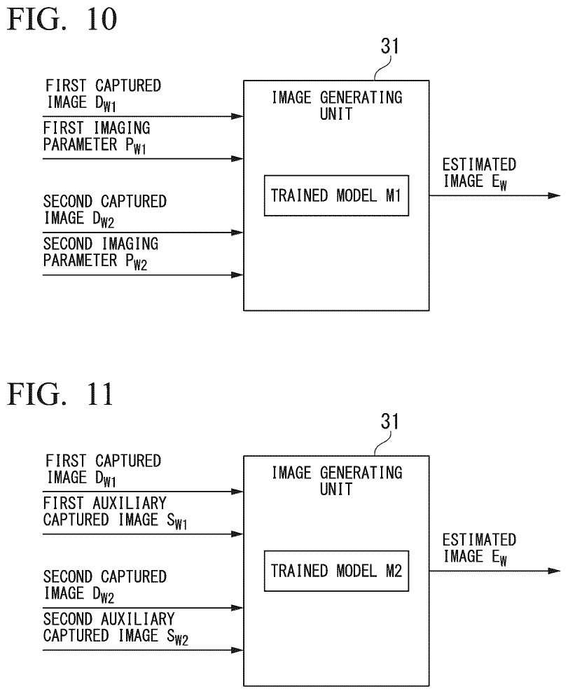

FIG. 10 is a diagram illustrating a trained model M1 which is a modified example of the trained model M.

The trained model M1 uses at least some of the imaging parameters P (for example, an analog gain) as input data. For example, the trained model MI receives a first imaging parameter P_{W1} which is an imaging parameter P when the first captured image D_{W1} is captured and a second imaging parameter P_{W2} which is an imaging parameter P when the second captured image D_{W2} is captured as additional inputs. By additionally using at least some of the imaging parameters P as inputs of the trained model M1, it is possible to enhance the estimation accuracy of the estimated image E_{W} in the image generating unit 31. The imaging parameters P are added as inputs to the training data which is used to train the trained model M1.

### MODIFIED EXAMPLE 2

FIG. 11 is a diagram illustrating a trained model M2 which is another modified example of the trained model M.

The trained model M2 uses an additional auxiliary captured image as input data. For example, the trained model M2 uses a first auxiliary captured image S_{W1} which is captured in a state in which the imaging parameters P other than the light intensity are not changed and the light intensity is set to zero immediately after the first captured image D_{W1} has been captured and a second auxiliary captured image S_{W2} which is captured in a state in which the imaging parameters P other than the light intensity are not changed and the light intensity is set to zero immediately after the second captured image D_{W2} has been captured as additional inputs. By adding auxiliary captured images captured with a light intensity set to zero as inputs of the trained model M2, it is possible to generate an estimated image E_{W} which is less likely to be affected by pattern noise of the imaging element 142 of the imaging unit 14 or the like. Particularly, when a CMOS image sensor having relatively more difficulty in noise reduction through correlation double sampling is used as the imaging element 142, it is possible to accurately generate the estimated image E_{W} by adding the auxiliary captured images captured with a light intensity set to zero as inputs. The auxiliary captured images are added as inputs to the training data used to train the trained model M2.

### MODIFIED EXAMPLE 3

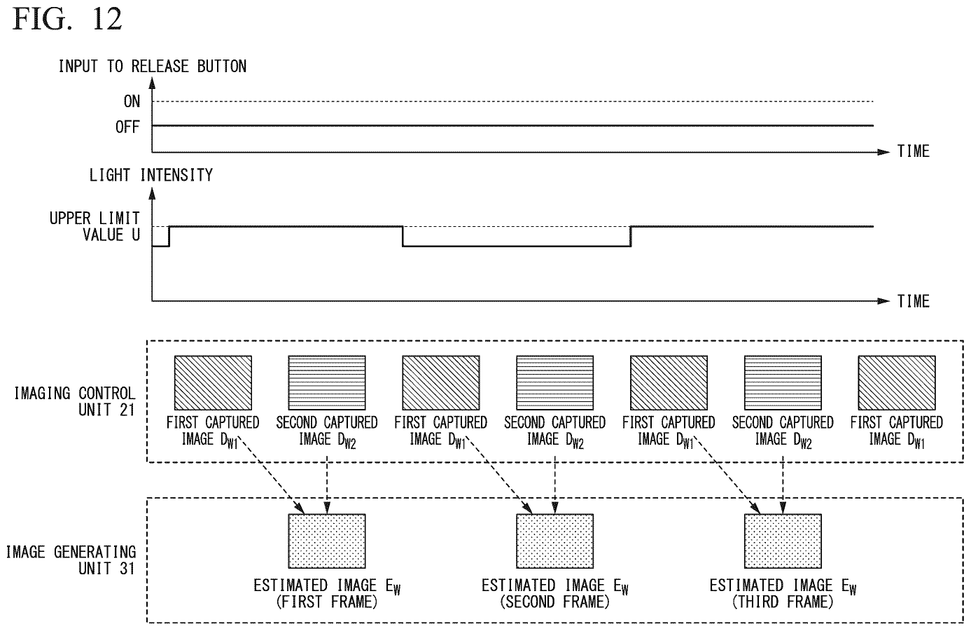

In the aforementioned embodiment, the endoscope system 100 infers one estimated image E_{W} by detecting an operation of pressing the release button 184. However, the endoscope system 100 may successively generate the estimated image E_{W} and generate a moving image with the successive estimated images E_{W} as frames regardless of whether an operation (ON) of pressing the release button 184 has been performed. FIG. 12 is a diagram illustrating a moving image (successive estimated images E_{W}) generated by the imaging generating unit 31. The endoscope system 100 generates one estimated image E_{W} by performing a series of Steps S110 to 170 and generates a moving image (successive estimated images E_{W}) by repeatedly performing the series of steps. The endoscope system 100 preferably has, for example, processing performance of generating the estimated image E_{W} at 30 FPS to generate a smooth moving image.

The endoscope system 100 adjusts the light intensity in Step S110 whenever one estimated image E_{w} is generated. Accordingly, the light intensity varies while generating a moving image. The light intensity preferably has a value which is equal to or less than the upper limit value U and close to the upper limit value U, but may be set to a value less than the upper limit value U based on the adjustment result of Step S110. The moving image generating sequence illustrated in FIG. 12 is a moving image generating sequence when the light intensity calculated in Step S120 is greater than the upper limit value U. When the light intensity calculated while generating a moving image is equal to or less than the upper limit value U, the endoscope system 100 generates a first captured image D_{W1} in Step S160 and uses the generated first captured image D_{W1} as one frame of the moving image.

With the endoscope system 100, it is possible to curb a light intensity of illumination light emitted from the illumination unit 15 to be equal to or less than the upper limit value U and to present to a user a moving image including successive estimated images E_{W} which are brighter than the first captured image D_{W1} and in which noise included in the image has the same degree as noise generated in the first captured image D_{W1} through the imaging signal processing.

### MODIFIED EXAMPLE 4

In the aforementioned embodiment, the illumination unit irradiates a subject with white light, but illumination light emitted from the illumination unit is not limited thereto. The illumination unit may irradiate the subject, for example, with special light which will be described later.

### Second Embodiment

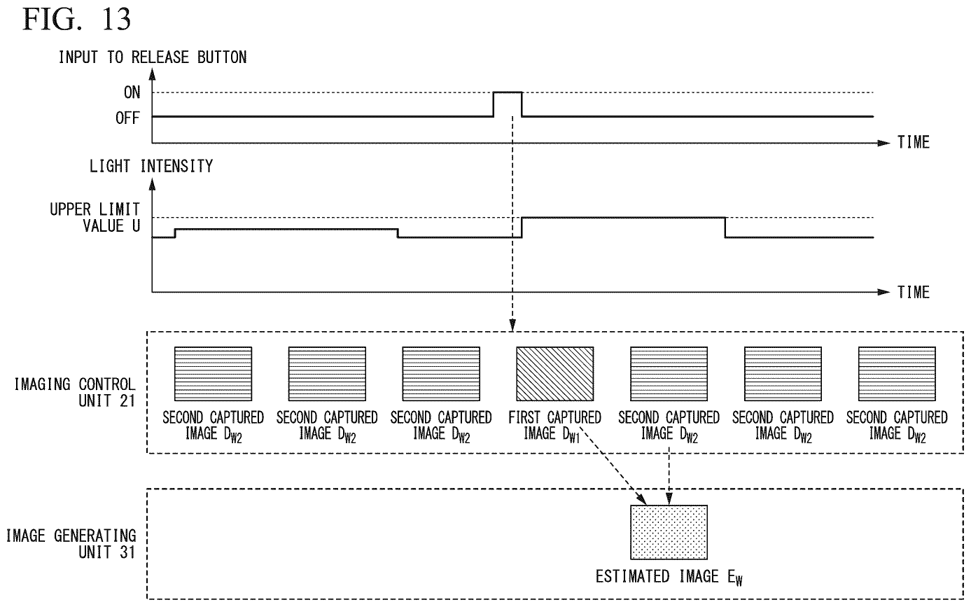

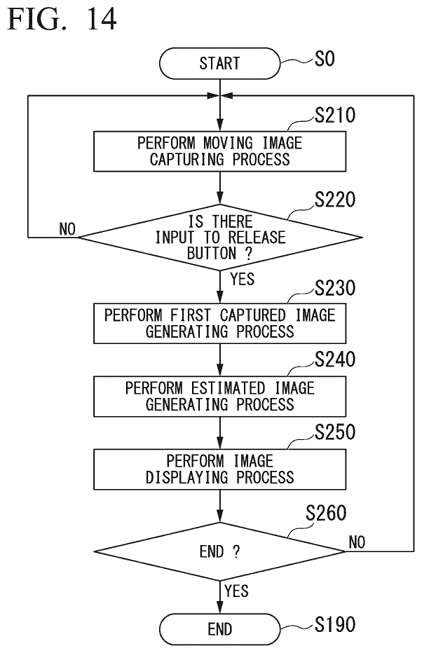

A second embodiment of the present disclosure will be described below with reference to FIGS. 13 and 14. An endoscope system 100B according to the second embodiment is different from the endoscope system 100 according to the first embodiment in only the control flow. In the following description, the same constituents as described above will be referred to by the same reference signs, and repeated description thereof will be omitted.

The endoscope system 100B displays a captured video (a moving image) on the display device 4 and generates a captured image (a still image) when the control unit 24 of the control device 2 detects a user's operation (ON) of pressing the release button 184. FIG. 13 is a diagram illustrating a captured image generated by the imaging control unit 21 and a still image (an estimated image E_{W}) generated by the image generating unit 31. Description will be continued with reference to a control flowchart for the endoscope system 100B illustrated in FIG. 14. When the endoscope system 100B is started, the endoscope system 100B performs Step S210.

### Step S210: Moving Image Generating Step

In Step S210, the control unit 24 of the control device 2 calculates imaging parameters P (which include a light intensity and an analog gain) optimal for imaging a subject and successively generates a second captured image D_{W2}. For example, the control unit 24 of the control device 2 generates the second captured images D_{W2} at 30 FPS. As illustrated in FIG. 13, the control unit 24 of the control device 2 adjusts the light intensity in a range equal to or less than the upper limit value U. The control unit 24 of the control device 2 displays the successively generated second captured images D_{W2} as a captured video (a moving image) on the display device 4.

### Step S220: Release Detecting Step

In Step S220, the control unit 24 of the control device 2 detects an operation of pressing the release button 184. Unless an operation of pressing the release button 184 is detected, the control unit 24 of the control device 2 continues to perform Step S210. When an operation of pressing the release button 184 is detected, the endoscope system 100B performs Step S230.

### Step S230: First Captured Image Generating Step

Similarly to Step S130 in the first embodiment, the control unit 24 of the control device 2 sets the light intensity which is an imaging parameter P to be equal to or less than the upper limit value U and generates a first captured image D_{W1}. For example, the control unit 24 generates the first captured image D_{W1} by temporarily decreasing the analog gain which is an imaging parameter P. The control unit 24 preferably sets the light intensity to a value which is equal to or less than the upper limit value U and closer to the upper limit value at the time of generating one first captured image D_{W1}. After having generated one first captured image D_{W1}, the control unit 24 causes the imaging control unit 21 to successively generate a second captured image D_{W2} and causes the display device 4 to display the successively generated second captured images D_{W2} as a captured video (a moving image). Then, the endoscope system 100B performs Step S240.

### Step S240: Estimated Image Generating Step

The image generating unit 31 of the image generation device 3 generates an estimated image E_{W} from the first captured image D_{W1} generated in Step S230 and the second captured images D_{W2} generated before and after the first captured image D_{W1} has been generated based on the trained model M. The image storage unit 33 records the estimated image E_{W} according to necessity. Then, the endoscope system 100B performs Step S250.

### Step S250: Image Displaying Step

The image display unit 32 of the image generation device 3 displays the estimated image E_{W} generated in Step S240 on the display device 4. The image display unit 32 of the image generation device 3 may simultaneously display the second captured images D_{W2} displayed as a moving image and the estimated image E_{W} on the display device 4. The image display unit 32 of the image generation device 3 may display the estimated image E_{W} on the display device 4 in a predetermined period in replacement of the second captured images D_{W2} displayed as a moving image. Then, the endoscope system 100B performs Step S260.

### Step S260: End Determining Step

In Step S260, the control unit 24 of the control device 2 determines whether display of a moving image is to end. When display of a moving image is not to end, the control unit 24 of the control device 2 performs Step S210 again.

Similarly to Modified Example 3, the endoscope system 100B may successively generate the estimated image E_{W} and generate a moving image using the successive estimated images E_{W} as frames.

With the endoscope system 100B according to the present embodiment, even when a still image is acquired while acquiring a captured video (a moving image), it is possible to curb an increase in temperature of the distal end portion 11 of the endoscope 1 and to provide a captured image (a still image) which is bright enough.

While the second embodiment of the present disclosure has been described above in detail with reference to the drawings, any specific configuration is not limited to this embodiment and includes design modification or the like without departing from the gist of the present disclosure. The constituents described in the aforementioned embodiment and the modified examples can be appropriately combined.

### MODIFIED EXAMPLE 5

In the aforementioned embodiment, the endoscope system 100B uses the successively generated second captured images D_{W2} as a captured video (a moving image) and generates an estimated image E_{W} as a captured image (a still image) using one first captured image D_{W1} generated to correspond to an operation of pressing the release button 184. However, generation of a captured video (a moving image) and a still image is not limited thereto. The endoscope system 100B may use the successively generated first captured images D_{W1} as a captured video (a moving image) and generates an estimated image E_{W} as a captured image (a still image) using one second captured image D_{W2} generated to correspond to an operation of pressing the release button 184.

### Third Embodiment

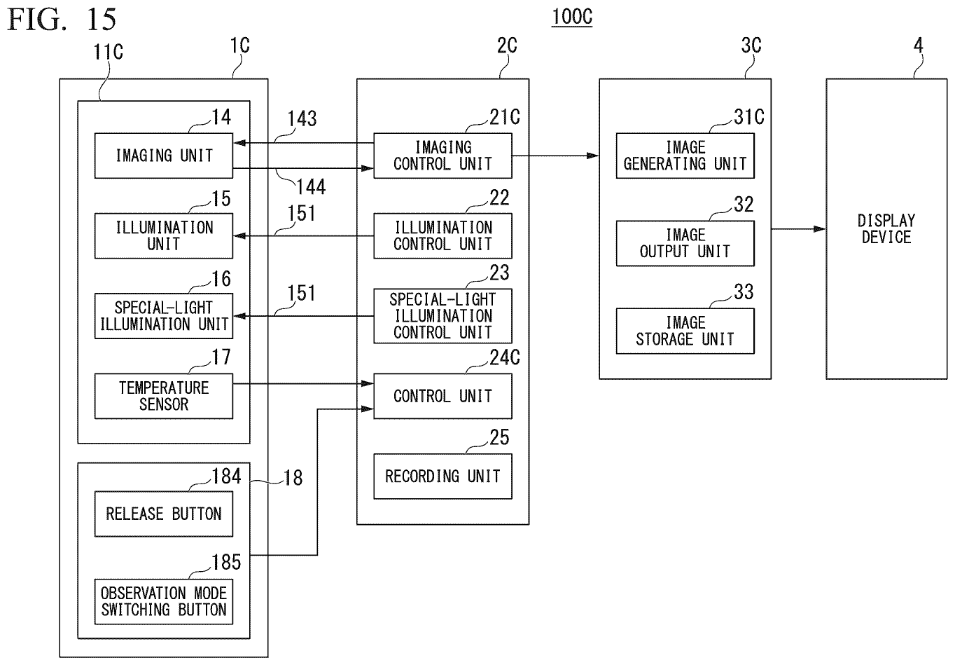

A third embodiment of the present disclosure will be described below with reference to FIGS. 15 to 17. An endoscope system 100C according to the third embodiment is different from the endoscope system 100 according to the first embodiment in that two types of observation modes can be used. In the following description, the same constituents as described above will be referred to by the same reference signs, and repeated description thereof will be omitted.

FIG. 15 is a functional block diagram of the endoscope system 100C.

The endoscope system 100C includes an endoscope 1C, a control device 2C, an image generation device 3C, and a display device 4.

The endoscope 1C is the same as the endoscope 1 according to the first embodiment except for a distal end portion 11C and a control device 2C. The distal end portion 11C of the endoscope 1C includes an imaging unit 14, an illumination unit 15, a special-light illumination unit 16, and a temperature sensor 17.

The special-light illumination unit 16 irradiates a subject with special light which is used in NBI (registered trademark: narrow band imaging), RDI (registered trademark: red dichromatic imaging), or the like. Here, special light emitted from the special-light illumination unit 16 is light in a wavelength band different from that of white light. Special light used in NBI is light in two wavelength bands of blue (wavelengths of 390 nm to 445 nm) and green (wavelengths of 530 nm to 550 nm) which are narrowed. Special light used in RDI is light in three wavelength bands of red, amber, and green which are narrowed.

The control device 2C is a device that controls the endoscope system 100C as a whole. The control device 2C includes an imaging control unit 21C, an illumination control unit 22, a special-light illumination control unit 23, a control unit 24C, and a recording unit 25.

The imaging control unit 21C has the same function as the imaging control unit 21 in the first embodiment and can additionally generate a special-light captured image (a special-light captured video) D_{S} by performing imaging signal processing on an imaging signal obtained by imaging a subject irradiated with special light based on the imaging parameters P.

The special-light illumination control unit 23 controls a light source of special light based on the imaging parameters P (such as a light intensity of special light) from the control unit 24 such that a light intensity of special light transmitted to the special-light illumination unit 16 is controlled.

The control unit 24C has the same function as the control unit 24 in the first embodiment and can additionally switch between two types of observation modes (a white-light observation mode and a special-light observation mode) based on an operation input to the observation mode switching button 185 or the like. In the white-light observation mode, the control unit 24C generates a captured image (a white-light captured image) D_{W} from an imaging signal obtained by imaging a subject irradiated with white light by the illumination unit 15. In the special-light observation mode, the control unit 24C generates a special-light captured image D_{S} from an imaging signal obtained by imaging a subject irradiated with special light by the special-light illumination unit 16.

The image generation device 3C includes an image generating unit 31C, an image display unit 32, and an image storage unit 33 as functional blocks.

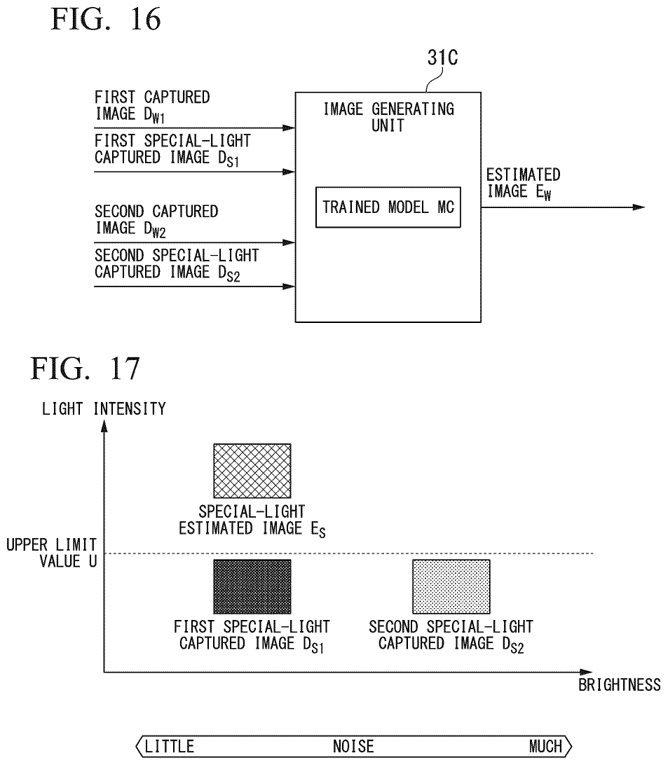

FIG. 16 is a diagram illustrating the image generating unit 31C.

The image generating unit 31C generates an estimated image E_{W} from a first captured image D_{W1}, a second captured image D_{W2}, a first special-light captured image D_{S1}, and a second special-light captured image D_{S2} input from the control device 2C based on a trained model MC.

FIG. 17 is a diagram illustrating a relationship between the first special-light captured image D_{S1} and second special-light captured image D_{S2}. The first special-light captured image D_{S1} is a special-light captured image in which a subject irradiated with special light with a light intensity equal to or less than the upper limit value U is imaged. The second special-light captured image D_{S2} is a special-light captured image in which a subject irradiated with special light with a light intensity equal to or less than the upper limit value U is imaged and which has more noise and higher brightness through imaging signal processing (the analog signal processing by the analog signal processor 212 and/or the digital signal processing by the digital signal processor 214) based on the imaging parameters P in comparison with the first special-light captured image D_{S1}.

The first special-light captured image D_{S1} and the second special-light captured image D_{S2} are preferably captured images in which a subject irradiated with a light intensity equal to or less than the upper limit value U and close to the upper limit value is imaged. By causing the light intensity to become closer to the upper limit value U, the first special-light captured image D_{S1} and the second special-light captured image D_{S2} become captured images with higher brightness and less noise. As a result, it is possible to enhance estimation accuracy of an estimated image E_{W}. Particularly, by reducing noise in the second special-light captured image D_{S2}, it is possible to efficiently enhance the estimation accuracy of an estimated image E_{W}.

The first special-light captured image D_{S1} is a special-light captured image which has been captured when a period T3 has elapsed after the first captured image D_{W1} has been captured. Since the period T3 is a very short period, the subject imaged in the first captured image D_{W1} and the first special-light captured image D_{S1} is substantially in the same state. The order of imaging of the first captured image D_{W1} and the first special-light captured image D_{S1} is not particularly limited.

The second special-light captured image D_{S2} is a special-light captured image which has been captured when a period T4 has elapsed after the second captured image D_{W2} has been captured. Since the period T4 is a very short period, the subject imaged in the second captured image D_{W2} and the second special-light captured image D_{S2} is substantially in the same state. The order of imaging of the second captured image D_{W2} and second special-light captured image D_{S2} is not particularly limited.

The trained model MC additionally uses the first special-light captured image D_{S1} and the second special-light captured image D_{S2} as inputs in comparison with the trained model M in the first embodiment. Special-light captured images are added as inputs to training data used to train the trained model Mc.

When the control unit 24C of the control device 2C detects an operator's operation of pressing the release button 184, the endoscope system 100C generates a first captured image D_{W1}, a second captured image D_{W2}, a first special-light captured image D_{S1}, and a second special-light captured image D_{S2} and generates an estimated image E_{W} from these four captured images.

Similarly to Modified Example 3, the endoscope system 100C may successively generate the estimated image E_{W} and generate a moving image using the successive estimated images E_{W} as frames. Similarly to the second embodiment, the endoscope system 100C may use the successively generated second captured images D_{W2} as a captured video (a moving image) and generate an estimated image E_{W} as a captured image (a still image) using the first captured image D_{W1}, the first special-light captured image D_{S1}, and the second special-light captured image D_{S2} generated to correspond to an operation of pressing the release button 184.

With the endoscope system 100C according to the present embodiment, it is possible to curb the light intensities of illumination light emitted from the illumination unit 15 and the special-light illumination unit 16 to be equal to or less than the upper limit value U and to present to a user an estimated image E_{W} which is brighter than the first captured image D_{W1} and in which noise included in the image has the same degree as noise generated in the first captured image D_{W1} through the imaging signal processing.

Special-light captured images acquired from an imaging signal obtained by imaging a subject irradiated with special light are added as inputs to the trained model MC. Since two types of captured images obtained by imaging a subject irradiated with two types of illumination light (white light and special light) have different characteristics, it is possible to appropriately enhance the estimation accuracy of an estimated image E_{W} by the image generating unit 31C.

While the third embodiment of the present disclosure has been described above in detail with reference to the drawings, any specific configuration is not limited to this embodiment and includes design modification or the like without departing from the gist of the present disclosure. The constituents described in the aforementioned embodiment and the modified examples can be appropriately combined.

### MODIFIED EXAMPLE 6

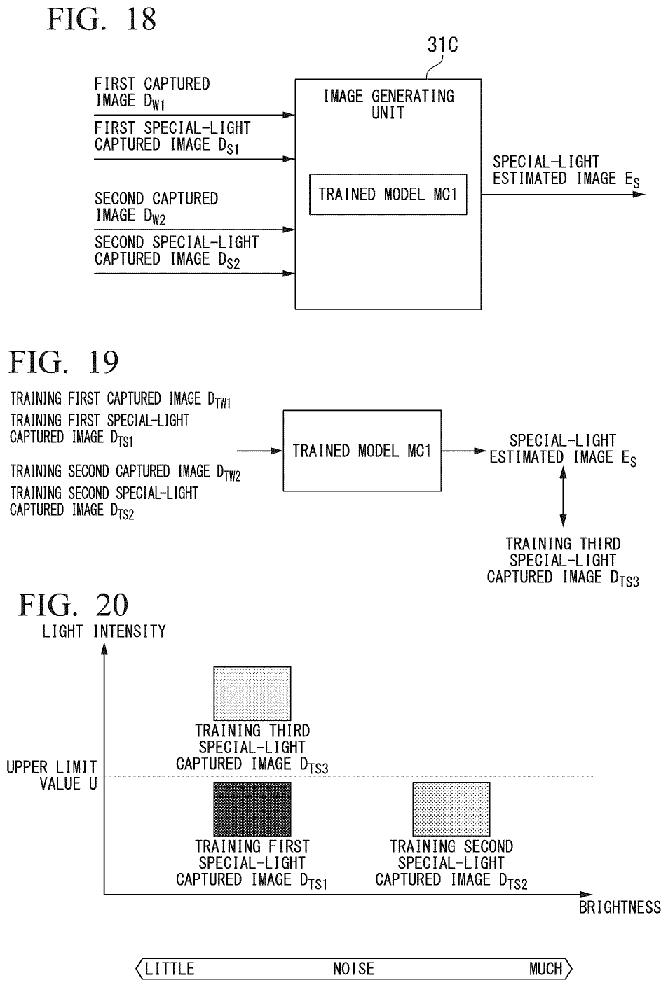

FIG. 18 is a diagram illustrating a trained model MC1 which is a modified example of the trained model MC.

The trained model MC1 outputs a special-light estimated image E_{S}. As illustrated in FIG. 17, the special-light estimated image E_{S} is an image which is brighter than the first special-light captured image D_{S1} and in which noise included in the image has the same degree as noise generated in the first special-light captured image D_{S1} through the imaging signal processing. The trained model MC1 may output both of the estimated image E_{W} and the special-light estimated image E_{S}.

FIG. 19 is a diagram illustrating generation of the trained model MC1.

Training data is an image group including a training first captured image D_{TW1}, a training first special-light captured image D_{TS1}, a training second captured image D_{TW2}, a training second special-light captured image D_{TS2}, and a training third special-light captured image D_{TS3} as a group of images.

FIG. 20 is a diagram illustrating training data which is used to generate the trained model MC1.

The training first special-light captured image D_{TS1} is a first special-light captured image D_{S1} prepared for learning. The training second special-light captured image D_{TS2} is a second special-light captured image D_{S2} prepared for learning.

The training third special-light captured image D_{TS3} is a captured image obtained by actually imaging a subject irradiated with special light with a light intensity greater than the upper limit value U. The training third special-light captured image D_{TS3} is an image in which noise generated through the imaging signal processing (the analog signal processing by the analog signal processor 212 and/or the digital signal processing by the digital signal processor 214) based on the imaging parameters P has the same degree as noise generated in the training first special-light captured image D_{TS1} through the imaging signal processing. For example, the training third special-light captured image D_{TS3} is an image in which the imaging parameters P other than the light intensity is substantially the same as the imaging parameters P of the training first special-light captured image D_{TS1}.

### MODIFIED EXAMPLE 7

In the aforementioned embodiment, the endoscope 1C uses two types of observation modes. When the endoscope 1C has three or more types of observation modes, three or more types of captured images may be able to be input to the trained model MC.

### Fourth Embodiment

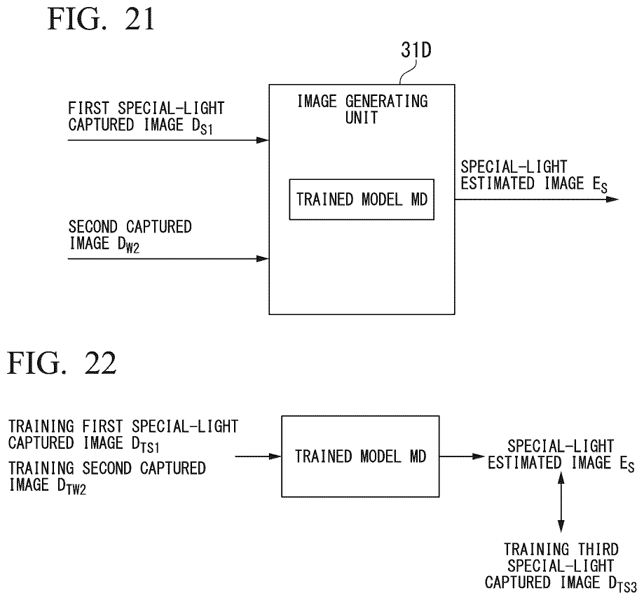

A fourth embodiment of the present disclosure will be described below with reference to FIGS. 21 and 22. An endoscope system 100D according to the fourth embodiment can use two types of observation modes similarly to the endoscope system 100C according to the third embodiment. In the following description, the same constituents as described above will be referred to by the same reference signs, and repeated description thereof will be omitted.

The endoscope system 100D is different from the endoscope system 100C according to the third embodiment, in that an image generating unit 31D is provided instead of the image generating unit 31C.

FIG. 21 is a diagram illustrating the image generating unit 31D.

The image generating unit 31D generates a special-light estimated image E_{S} from the first special-light captured image D_{S1} and the second special-light captured image D_{S2} input from the control device 2C based on the trained model MD.

FIG. 22 is a diagram illustrating generation of the trained model MD.

Training data is an image group including a training first special-light captured image D_{TS1}, a training second special-light captured image D_{TS2}, and a training third special-light captured image D_{TS3} as a group of images. The training data preferably includes images captured under various conditions as many as possible.

The trained model MD generates the special-light estimated image E_{S} based on the training first special-light captured image D_{TS1} and the training second captured image D_{TW2} which are input. The trained model MD learns parameters of the trained model MD such that a difference between the special-light estimated image E_{S} and the training third special-light captured image D_{TS3} which are output decreases.

The training third special-light captured image D_{TS3} is a captured image which is captured after a period T5 has elapsed after the training first special-light captured image D_{TS1} and the training second captured image D_{TW2} have been captured. Since the period T5 is a very short period, the subject imaged in the training first special-light captured image D_{TS1}, the training second captured image D_{TW2}, and the training third special-light captured image D_{TS3} is substantially in the same state. The order of imaging of the training first special-light captured image D_{TS1}, the training second captured image D_{TW2}, and the training third special-light captured image D_{TS3} is not particularly limited.

When the control unit 24C of the control device 2C detects an operator's operation of pressing the release button 184, the endoscope system 100D generates the first special-light captured image D_{S1} and the second captured image D_{W2} and generates the special-light estimated image E_{S} from these two captured images.

Similarly to Modified Example 3, the endoscope system 100D may successively generate the special-light estimated image E_{S} and generate a moving image including the successive special-light estimated images E_{S} as frames. Similarly to the second embodiment, the endoscope system 100D may use the successively generated second captured images D_{W2} as a captured video (a moving image) and generate the special-light estimated image E_{S} as a captured image (a still image) using the first special-light captured image D_{S1} generated corresponding to the operation of pressing the release button 184.

With the endoscope system 100D according to the present embodiment, it is possible to curb the light intensities of illumination light emitted from the illumination unit 15 and the special-light illumination unit 16 to be equal to or less than the upper limit value U and to present to a user a special-light estimated image E_{S} which is brighter than the first special-light captured image D_{S1} and in which noise included in the image has the same degree as noise generated in the first special-light captured image D_{S1} through the imaging signal processing. The endoscope system 100D requires a smaller number of captured images to generate an estimated image in comparison with the endoscope system 100C according to the third embodiment. Accordingly, it is possible to further enhance a frame rate when a moving image including the estimated image as a frame is generated as in Modified Example 3.

A special-light captured image may be darker than a white-light captured image according to a type of special light. For example, a special-light captured image acquired by NBI is darker than a white-light captured image. Special light used in NBI is more likely to be absorbed by human tissue which is a subject than white light and is less likely to be reflected by human tissue. Accordingly, if the emitted light intensity is the same, the light intensity received by the imaging element 142 is weaker when a subject is irradiated with special light than when the subject is irradiated with white light. Accordingly, when a special-light captured image is acquired by NBI, it is difficult to provide a special-light captured image which is bright enough in usage (for example, parallel display of a white-light captured image and a special-light captured image) in which a white-light captured image and a special-light captured image are combined for use. However, the endoscope system 100D can generate and provide a special-light estimated image E_{S} brighter than the first special-light captured image D_{S1} from the first special-light captured image D_{S1} and the second captured image D_{W2}.

While the fourth embodiment of the present disclosure has been described above in detail with reference to the drawings, any specific configuration is not limited to this embodiment and includes design modification or the like without departing from the gist of the present disclosure. The constituents described in the aforementioned embodiment and the modified examples can be appropriately combined.

### MODIFIED EXAMPLE 8

In the aforementioned embodiment, the imaging unit 14 generating an imaging signal is provided in the endoscope 1, and the image generation device 3 or 3C generates an estimated image E based on a captured image acquired from the imaging unit 14 of the endoscope 1. However, a source of the captured signal in the image generation device is not limited to the endoscope 1. The image generation device may generate the estimated image E based on a captured image acquired from another imaging device such as a camera, a video camera, an industrial endoscope, a microscope, a robot having an image recognizing function, or a mobile device such as a smartphone, a mobile phone, a smart watch, a table terminal, or a notebook PC.

### MODIFIED EXAMPLE 9

In the aforementioned embodiment, an LED is exemplified as a light source of the illumination unit 15, the illumination control unit 22, or the like, but the light source is not limited to an LED. For example, the light source may be a laser source including a laser diode, an electric lamp of organic EL, xenon, halogen, or the like, a combination thereof, or a combination of an optical element such as a fluorescent substance having a wavelength conversion function therewith.

### MODIFIED EXAMPLE 10

In the aforementioned embodiment, the imaging parameter P is a light intensity or a gain, but the imaging parameter P is not limited thereto. The imaging parameter P may be a spectral distribution, a diaphragm value (F-value), a shutter speed, a frame rate of a moving image, an optical magnification, a digital magnification, or a grayscale, pixel size, or resolution (DPI) in a process in which the imaging control unit 21 generates a captured image (a captured video) from an imaging signal captured by the imaging element 142. The imaging parameter P may include an organ type (the throat, the stomach, or the duodenal) of a subject. The organ type may be determined from a captured image by a model such as a machine learning model, may be determined from an insertion distance of the distal end portion 11 of the endoscope 1 into the body, or may be input to the image generation device 3 by an operator.

Programs in the embodiments and the modified examples thereof may be recorded on a computer-readable recording medium, and a computer system may be caused to read and execute the programs recorded on the recording medium. The “computer system” includes an OS or hardware such as peripherals. The “computer-readable recording medium” is a portable medium such as a flexible disk, a magneto-optical disc, a ROM, or a CD-ROM or a storage device such as a hard disk incorporated into a computer system. The “computer-readable recording medium” may include a medium that dynamically hold a program for a short time such as a communication line when the program is transmitted via a network such as the Internet or a communication line such as a telephone line or a medium that holds a program for a predetermined time such as a volatile memory in a computer system serving as a server or a client in that case. The program may be a program for realizing some of the aforementioned functions or may be a program for realizing the aforementioned functions in combination with another program stored in advance in the computer system.

What is claimed is:

1. An endoscope system comprising:
an endoscope including an illumination unit that irradiates a subject with illumination light and an imaging unit that images the subject;
a control device that generates a captured image by performing imaging signal processing on an imaging signal acquired from the imaging unit based on an imaging parameter; and
an image generation device,
wherein the control device generates a first captured image in which the subject irradiated with a light intensity equal to or less than an upper limit value is imaged and a second captured image in which the subject irradiated with a light intensity equal to or less than the upper limit value is imaged and which is brighter than the first captured image, and
wherein the image generation device generates an estimated image obtained by estimating an image in which the subject irradiated with a light intensity greater than the upper limit value is imaged from the first captured image and the second captured image.

2. The endoscope system according to claim 1, further comprising a display device displaying the captured images,
wherein the control device successively generates the second captured image, causes the display device to display the successive second captured images as a moving image, and generates the first captured image as a still image, and
wherein the image generation device generates the estimated image from the first captured image and the second captured images generated before and after the first captured image has been generated.

3. The endoscope system according to claim 1, wherein the image generation device generates the estimated image based on a trained model having learned a relationship between the first captured image and the second captured image and the estimated image in advance through machine learning.

4. The endoscope system according to claim 3, wherein the trained model is a model having been trained through machine learning using the first captured image prepared for training, the second captured image prepared for training, and a third captured image as training data, and
wherein the third captured image is a captured image in which the subject irradiated with a light intensity greater than the upper limit value is imaged and in which noise generated through the imaging signal processing has the same degree as noise generated in the first captured image through the imaging signal processing.

5. The endoscope system according to claim 3, wherein the trained model uses at least some of the imaging parameter as input data.

6. The endoscope system according to claim 1, wherein noise included in the estimated image has the same degree as noise generated through the imaging signal processing in the first captured image.

7. The endoscope system according to claim 1, wherein there is more noise generated through the imaging signal processing in the second captured image than noise generated through the imaging signal processing in the first captured image.

8. The endoscope system according to claim 7, wherein the imaging signal processing includes an amplification process on the imaging signal, and
wherein a gain of the imaging parameter for generating the second captured image is greater than a gain of the imaging parameter for generating the first captured image.

9. The endoscope system according to claim 1, wherein the control device acquires a temperature of the illumination unit and determines the upper limit value of the light intensity such that the temperature is equal to or less than a predetermined temperature.

10. The endoscope system according to claim 1, wherein the endoscope further includes a special-light illumination unit emitting special light in a wavelength range different from that of the illumination light emitted from the illumination unit,
wherein the control device is able to generate a special-light captured image by performing imaging signal processing based on the imaging parameter on an imaging signal obtained by imaging the subject irradiated with the special light,
wherein the control device generates a first special-light captured image which is a special-light captured image in which the subject irradiated with the special light with a light intensity equal to or less than the upper limit value is imaged and a second special-light captured image which is a special-light captured image in which the subject irradiated with the special light with a light intensity equal to or less than the upper limit value is imaged and which is brighter than the first special-light captured image, and
wherein the image generation device generates the estimated image from the first captured image, the second captured image, the first special-light captured image, and the second special-light captured image.

11. The endoscope system according to claim 10, wherein the image generation device generates the estimated image based on a trained model having learned a relationship between the first captured image, the second captured image, the first special-light captured image, the second special-light captured image, and the estimated image in advance through machine learning.

12. The endoscope system according to claim 1, wherein the endoscope further includes a special-light illumination unit emitting special light in a wavelength range different from that of the illumination light emitted from the illumination unit,
wherein the first captured image is an image which is generated by performing imaging signal processing on an imaging signal obtained by imaging the subject irradiated with the special light by the special-light illumination unit,
wherein the second captured image is an image which is generated by performing imaging signal processing on an imaging signal obtained by imaging the subject irradiated with the illumination light by the illumination unit, and
wherein the estimated image is an image which is obtained by estimating an image in which the subject irradiated with the special light with a light intensity greater than the upper limit value is imaged from the first captured image and the second captured image.

13. The endoscope system according to claim 12, wherein the image generation device generates the estimated image based on a trained model having learned a relationship between the first captured image and the second captured image and the estimated image in advance through machine learning.

14. The endoscope system according to claim 13, wherein the trained model is a model having been trained through machine learning using the first captured image prepared for training, the second captured image prepared for training, and a third captured image, and
wherein the third captured image is a captured image in which the subject irradiated with the special light with a light intensity greater than the upper limit value is imaged and in which noise generated through the imaging signal processing has the same degree as noise generated in the first captured image through the imaging signal processing.

15. The endoscope system according to claim 1, wherein the image generation device successively generates the estimated image and generates a moving image having the successive estimated images as frames.

16. An image generation device for acquiring a captured image which is generated by performing imaging signal processing based on an imaging parameter on an imaging signal acquired from an imaging unit imaging a subject, the image generation device performing:
acquiring a first captured image in which the subject irradiated with a light intensity equal to or less than an upper limit value is imaged and a second captured image in which the subject irradiated with a light intensity equal to or less than the upper limit value is imaged and which is brighter than the first captured image; and
generating an estimated image obtained by estimating an image in which the subject irradiated with a light intensity greater than the upper limit value is imaged from the first captured image and the second captured image.

17. The image generation device according to claim 16, wherein the image generation device generates the estimated image based on a trained model having learned a relationship between the first captured image and the second captured image and the estimated image in advance through machine learning.

18. The image generation device according to claim 17, wherein the trained model is a model having been trained through machine learning using the first captured image prepared for training, the second captured image prepared for training, and a third captured image as training data, and
wherein the third captured image is a captured image in which the subject irradiated with a light intensity greater than the upper limit value is imaged and in which noise generated through the imaging signal processing has the same degree as noise generated in the first captured image through the imaging signal processing.

19. The image generation device according to claim 17, wherein the trained model uses at least some of the imaging parameter as input data.

20. The image generation device according to claim 16, wherein noise included in the estimated image has the same degree as noise generated through the imaging signal processing in the first captured image.

21. The image generation device according to claim 16, wherein there is more noise generated through the imaging signal processing in the second captured image than noise generated through the imaging signal processing in the first captured image.

22. The image generation device according to claim 21, wherein the imaging signal processing includes an amplification process on the imaging signal, and
wherein a gain of the imaging parameter for generating the second captured image is greater than a gain of the imaging parameter for generating the first captured image.

23. An image generation method comprising:
acquiring a first captured image which is generated by performing imaging signal processing on an imaging signal acquired by imaging a subject irradiated with a light intensity equal to or less than an upper limit value and a second captured image which is generated by performing imaging signal processing on an imaging signal acquired by imaging the subject irradiated with a light intensity equal to or less than the upper limit value and which is brighter than the first captured image; and
generating an estimated image obtained by estimating an image in which the subject irradiated with a light intensity greater than the upper limit value is imaged from the first captured image and the second captured image.

24. The image generation method according to claim 23, wherein the estimated image is generated based on a trained model having learned a relationship between the first captured image and the second captured image and the estimated image in advance through machine learning.

25. The image generation method according to claim 24, wherein the trained model is a model having been trained through machine learning using the first captured image prepared for training, the second captured image prepared for training, and a third captured image as training data, and
wherein the third captured image is a captured image in which the subject irradiated with a light intensity greater than the upper limit value is imaged and in which noise generated through the imaging signal processing has the same degree as noise generated in the first captured image through the imaging signal processing.

26. An image generation device comprising a processor configured to perform:
generating a first captured image in which a subject irradiated with a first light intensity is imaged based on a first imaging parameter;
generating a second captured image in which the subject irradiated with the first light intensity is imaged based on a second imaging parameter; and
generating an estimated image based on the first captured image and the second captured image,
wherein the second captured image is an image with a higher luminance value than the first captured image, and
wherein the estimated image is an image which is obtained by estimating the subject irradiated with a second light intensity greater than the first light intensity.

27. The image generation device according to claim 26, wherein the estimated image is generated based on a trained model having learned a relationship between the first captured image and the second captured image and the estimated image in advance through machine learning.

28. The image generation device according to claim 26, wherein the imaging parameter is for an amplification process on the imaging signal, and
wherein a gain of the second imaging parameter is greater than a gain of the first imaging parameter.

29. The image generation device according to claim 28, wherein the second captured image generated using the second imaging parameter includes more noise than noise included in the first captured image generated using the first imaging parameter.

30. The image generation device according to claim 26, wherein the first light intensity is a light intensity at which a temperature of an illumination unit irradiating the subject with illumination light is equal to or less than a predetermined temperature.

31. The image generation device according to claim 30, wherein the second light intensity is a light intensity at which the temperature of the illumination unit is greater than the predetermined temperature.
